(19) United States
(12) Patent Application Publication
(10) Pub. No.: US 2009/0012476 A1
(43) Pub. Date: Jan. 8, 2009

(54) HEMOSTASIS VALVE FOR A CATHETER INTRODUCER

(75) Inventor: David George Catlin, Heath, TX (US)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: GALT MEDICAL CORPORATION, Garland, TX (US)

(21) Appl. No.: 11/773,808

(22) Filed: Jul. 5, 2007

(51) Int. Cl. A61M 39/06

(52) U.S. Cl. 604/167.04

(57) ABSTRACT

A catheter introducer valve comprising a body having an upper surface and a lower surface; a rim having a portion thereof extending beyond the upper surface and a portion thereof extending beyond the lower surface; a substantially conical depression formed in the body extending from an outer portion proximate to the upper surface and tapering toward an inner portion; an aperture formed in the body coaxial with the conical depression, the aperture having a conical first portion, a second portion comprising a first slit coaxial with the first portion and extending from the conical depression inner portion into the body, a third portion comprising a slit flaring outward from and at least partially intersecting the aperture first portion toward the bottom surface; a first thickened portion associated with the bottom surface; and, a second thickened portion associate with the bottom surface and generally perpendicular to the first thickened portion.

### FIELD OF THE INVENTION

The present invention generally relates to introducer sheaths for use in medical procedures requiring vascular and nonvascular access. More specifically, the present invention relates to hemostasis gasket valves for use in introducer sheaths and other medical devices.

### BACKGROUND OF THE INVENTION

Intravascular catheters such as angiographic or angioplasty catheters are introduced into an artery or vein of a patient. After such an introduction, the catheter is advanced and maneuvered through the arteriovenous system to a desired site, such as an area of arterial stenosis or an area from which the x-ray contrast media is injected into the system. Such a catheter may be placed by first inserting a hollow needle with a trocar through the skin into the lumen of the desired blood vessel. Following this, a guidewire is passed through the needle and advanced up the artery or vein toward the desired area. The needle can then be removed, leaving the guidewire in the vessel.

Following this, a catheter introducer, typically comprising a tubular sheath and a removable, hollow stylet or dilator unit, may be advanced together over the guidewire into the vessel. Then, the guidewire and the dilator unit may be removed, leaving only the sheath of the catheter introducer member present in the vessel. Then, desired catheters can be advanced through the sheath into the vessel, particularly an artery, while reliably maintaining a seal against blood loss as the catheter is advanced, and also between the times that a catheter is being advanced or maintained in the blood vessel.

The sheath of the catheter introducer typically carries a hub, which has a hemostasis valve on its end distal from the patient, so as to avoid uncontrolled bleeding and air embolism. The dilator unit, and then respective catheters as desired, pass through this hemostasis valve. In several conventional valve designs, the typically elastomeric partition of the valve is carried by the hub and defines a special slit or septum which is particularly adapted to provide a fluid-tight seal about an advancing catheter which passes through the catheter introducer. The special slit should lower the frictional resistance to catheter advancement that is provided over a conventional, straight slit.

What is needed is a slit configuration that provides both improved sealing properties, as well as being resistant to movement or tearing in the hub and relatively easy to manufacture and assemble into a catheter introducer.

### SUMMARY OF THE INVENTION

The present invention provides, in one exemplary embodiment, a sealable valve for a catheter introducer, the valve comprising a body having an upper surface and a lower surface; a rim having a portion thereof extending beyond the upper surface and a portion thereof extending beyond the lower surface; a first slot formed in the rim portion extending beyond the upper surface; a second slot formed in the rim portion extending beyond the lower surface; a substantially conical depression formed in the body extending from an outer portion proximate to the upper surface and tapering toward an inner portion; an aperture formed in the body coaxial with the conical depression, the aperture having a first portion having a generally conical shape and tapering from the upper surface toward and to generally midway between the center of the conical depression and the lower surface, a second portion comprising a first slit coaxial with the first portion and extending from the conical depression inner portion into the body, a third portion comprising a slit flaring outward from and at least partially intersecting the aperture first portion toward the bottom surface; a first thickened portion associated with the bottom surface, the first thickened portion containing at least a portion of the aperture third portion slit; and, a second thickened portion associate with the bottom surface and generally perpendicular to the first thickened portion.

Other features of the present invention will become apparent upon reading the following detailed description of embodiments of the invention, when taken in conjunction with the appended claims.

### BRIEF DESCRIPTION OF THE DRAWINGS

The invention is illustrated in the drawings in which like reference characters designate the same or similar parts throughout the figures of which:

FIG. 1 is a cross-section view of one exemplary embodiment of a catheter introducer having the improved hemostasis valve of the present invention.

FIG. 2A is a detail of the lower portion of the exemplary embodiment of the hemostasis valve of FIG. 1.

FIG. 2B is a cutaway detail of the lower portion of the exemplary embodiment of the hemostasis valve taken along the line A-A of FIG. 2A.

FIG. 2C is a side view of the valve of FIG. 2A taken along line A-A of FIG. 2A.

FIG. 2D is a bottom view of the valve similar to FIG. 2C, rotated 90 degrees.

FIG. 2E is a side view of the valve of FIG. 2D taken along lines C-C of FIG. 2D.

FIG. 3A is a detail of the upper face of the exemplary embodiment of the hemostasis valve of FIG. 1.

FIG. 3B is a cutaway side detail of the lower portion of the exemplary embodiment of the hemostasis valve taken along the line B-B of FIG. 3A.

FIG. 3C is a side view of the valve of FIG. 3A.

FIG. 4A is a cross-section view of the exemplary embodiment of the valve of FIG. 1, showing details of the bottom slit.

FIG. 4B is a cross-section view of the exemplary embodiment of the valve of FIG. 1, showing details of the top slit turned 90 degrees.

FIG. 5 is a perspective detailed view showing the upper and lower key/slot combinations.

FIG. 6 is a perspective detailed view of an alternative embodiment showing a valve having only a lower key/slot combination.

FIG. 7 is a perspective detailed view of an alternative embodiment showing a valve having no lower key/slot combination.

FIG. 8 is a side elevational cutaway view of the embodiment of FIG. 7.

### DESCRIPTION OF THE PREFERRED EMBODIMENTS

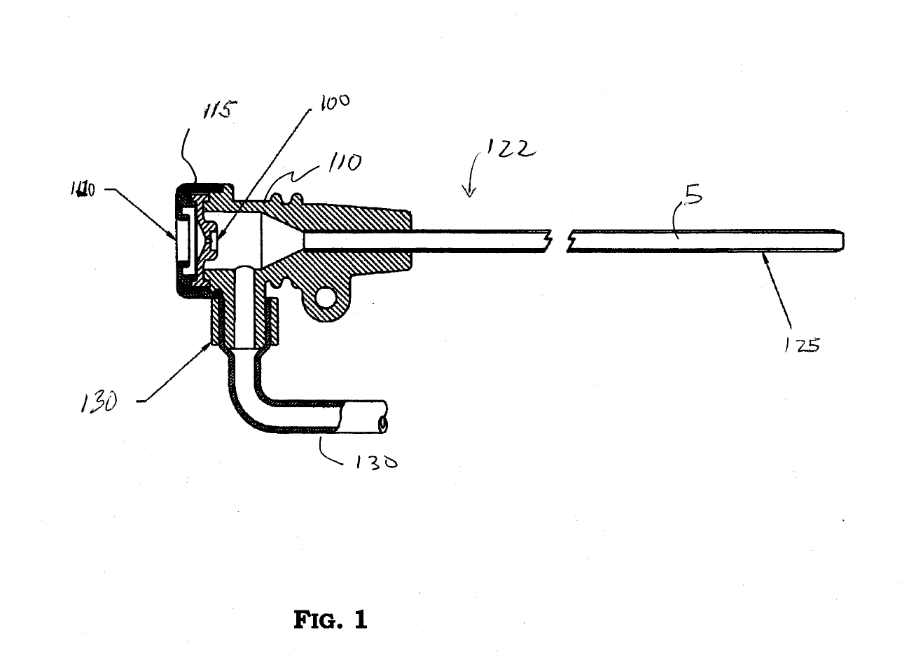

Referring to the drawings, FIG. 1 shows a cross-sectional view of a conventional catheter sheath introducer 5, adapted to receive an inner catheter (not shown, but known to those skilled in the art) for insertion into the vascular system of a patient.

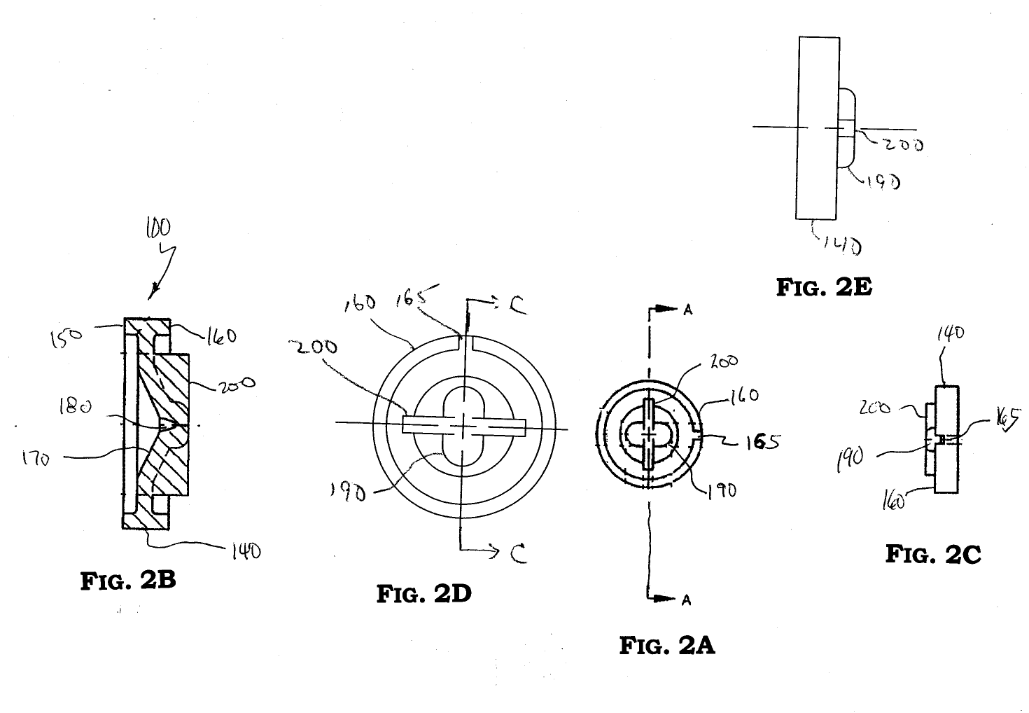

In one exemplary embodiment of the present invention an elastomeric hemostasis valve 100 is positioned in a hub 110. The hub 110 has a cap 115 that is attached to the hub 110 and holds the valve 100 in place. The cap 115 may be attached by screwing, snap fit, friction fit, welding, gluing or other means known to those skilled in the art. As described hereinbelow, the cap 115 has one or more keys (See FIGS. 2A and 2C) to engage the valve 100 and hold it firmly in place. The cap 115 of the hub 110 has an entrance hole 120 for insertion of a needle, dilator, catheter, guidewire, or the like, through the hole 120 and thus through the hemostasis valve 100. The introducer 5 has a sheath 125 and also an optional side port 130 for flushing the system.

The valve 100 is made of any biologically inert elastomeric material, preferably a silicone rubber having a Shore “A” durometer in the range of about 30-60, preferably about 50, and an elongation of up to about 1,400, more preferably, about 800. The valve 100 is preferably constructed by molding the material. The valve 100 has an upper face 102 and a lower face 104. The valve 100 may be lubricated with commercially available medical grade silicone or other lubricant known to those skilled in the art.

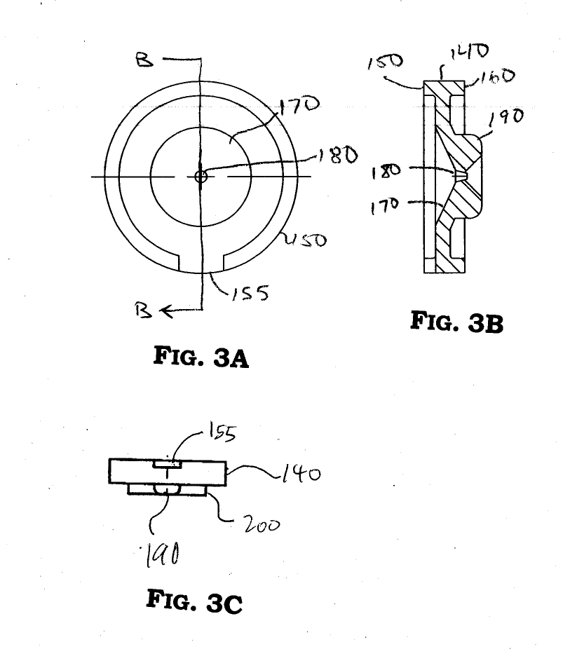

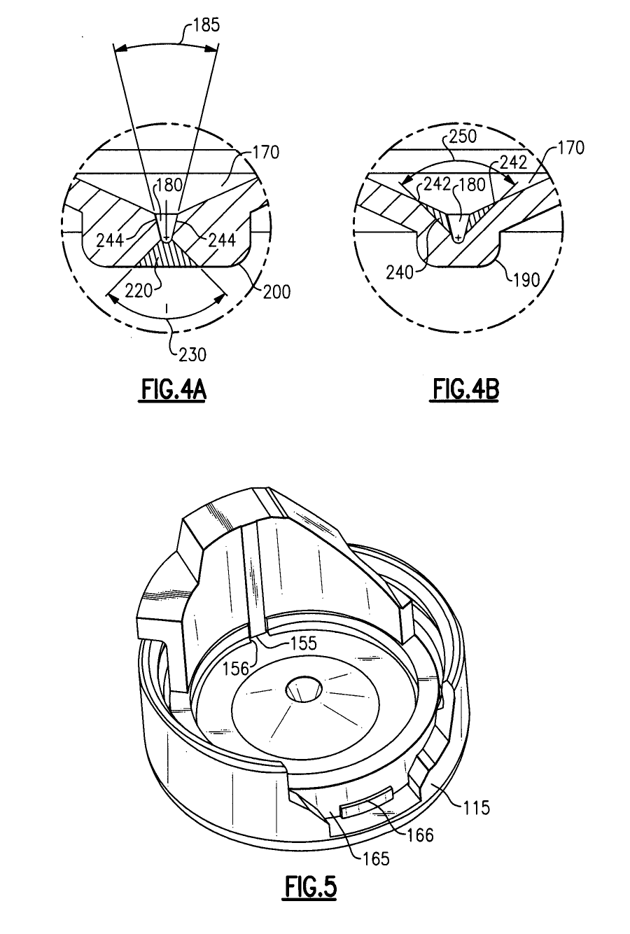

The hub 110 and cap 115 capture the valve 100 by its peripheral rim 140. FIGS. 2B and 3B show the peripheral rim 140 of the valve 100 in more detail.

Preferably, the peripheral rim 140 of the valve 100 has an upper C-shaped rim or lip 150 and a lower C-shaped rim or lip 160, as shown in FIG. 2B. The C-shapes 150, 160 of the peripheral rim 140 define an upper slot or opening 155 and a lower slot or opening 165, as shown in FIGS. 2A and 3A. The upper and lower openings 155, 165 are engageable with mating keys 156, 166 or protrusions in the hub 110 and cap 115, as shown in FIG. 5. The keys 155, 166 assist in locating the top and bottom of the valve. Alternatively, rather than a slot 155 or 165, a recessed or grooved portion can be used which contacts a complementary set of ribs or bumps rather than the mating keys 156, 166. Alternatively, the valve 100 may not have the flanges 150 and 160 and have a flat rim 140, in which case the catheter 115 is adapted to fit the valve 100 accordingly (as shown in FIG. 8) and the valve is held in place by axial load pressure.

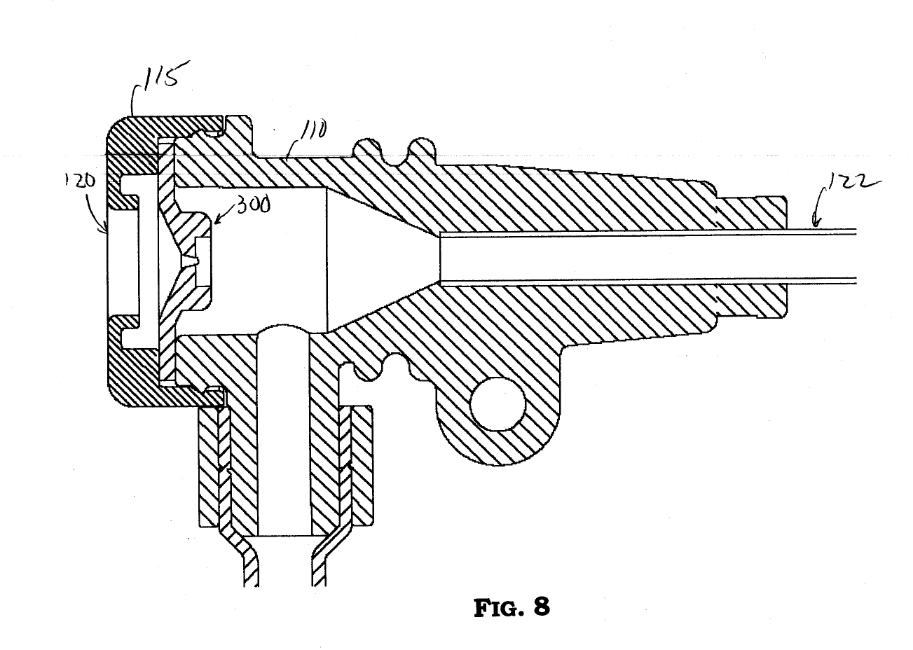

FIG. 6 shows an alternative exemplary embodiment of the keys and slots described with respect to FIG. 5. In the embodiment shown in FIG. 6 a single lower slot 165 and single lower key 166 is used. It is to be understood that alternatively, the embodiment shown in FIG. 5 can be modified to provide a single upper key 155 and upper slot 156 and no lower slot 165 or lower key 166.

FIGS. 7 and 8 show a further alternative exemplary embodiment of the present invention in which no keys or slots are used. Furthermore, in this particular embodiment the valve 300 does not have the flanges 150 or 160. Other than the elimination of keys and slots and the flanges, the remaining elements and aspects of the valve 300 are substantially the same as described with respect to valve 100.

FIGS. 2A-2E shows details of one exemplary embodiment of the valve 100, particularly the lower C-shaped lip 160 that is a part of the peripheral rim 140. The lower C-shaped lip 160 defines an opening 165 (see FIG. 2C) for engaging a key 166 in the hub 110. The valve 100 has a conical entrance 170 on its upper or distal face (the face toward the cap 115) tapering from the upper face 102 toward the center. The conical entrance 170 preferably has a total angle of about 130 degrees, though other angles can be used. At the center of the conical entrance 170 is a conical hole 180. The conical hole 180 has sides that preferably define an angle 185 of approximately 27 degrees, though other angles can be used, but in any case, is an angle smaller than that defined by the sides of the conical entrance 170. Alternatively, the conical entrance 170 may be curved (convex or concave), as desired. Furthermore, alternatively, the conical entrance 170 may extend to proximate to the edge of the rim 140.

The lower or proximal face 104 of the valve 100 (see FIG. 2A) has a first thickened portion 190 and a second thickened portion 200 oriented substantially perpendicular to the first thickened portion 190. FIG. 2C shows a cross-section view taken along line A-A of the second thickened portion 200. The thickened portions 190, 200 provide elastic restoring forces that assist the closing of the valve 100 about a needle, dilator or other object.

Slits as described hereinbelow may be created by a slitting apparatus.

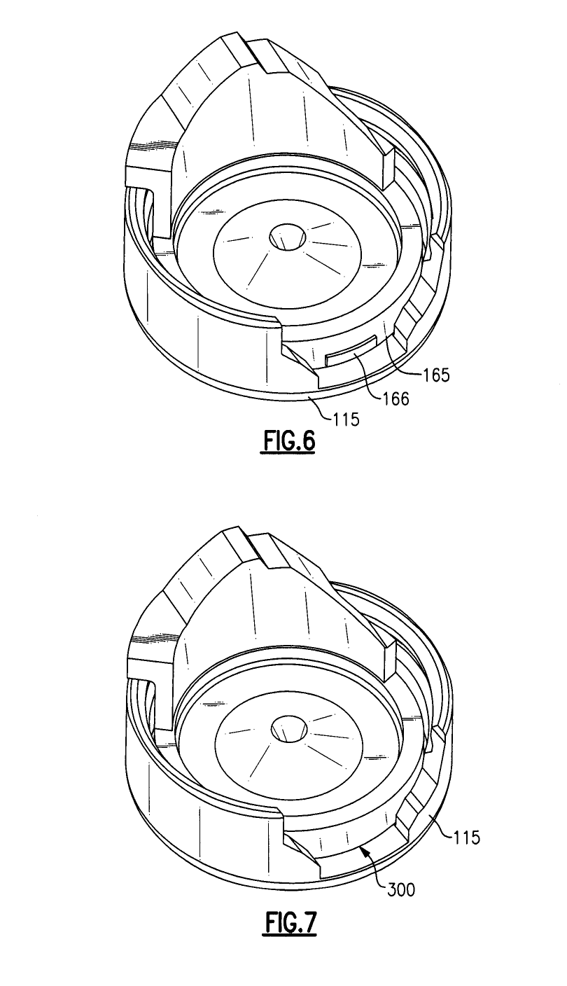

FIGS. 4A and 4B show details of the openings 155, 165 in the valve 100. FIG. 4A shows the conical hole 180 that captures the point of a needle, dilator or other object. Intersecting the deepest part of the conical hole 180 is a lower slit 220. The lower slit 220 is aligned with the second thickened portion 200. The lower slit 220 is cut into the thickened portion 19, preferably having a rhombohedral or trapezoidal shape. The angle 230 (shown as dashed lines) defined by the sides of the lower slit 220 is approximately 90 degrees in the preferred embodiment, although other angles can be used. In an alternative exemplary embodiment, the sides of the lower slit 220 can be cut into the second thickened portion 200, preferably having a rectangular shape.

FIG. 4B shows an upper slit 240 in the conical hole 180 viewed from upper face of the valve 100. The upper slit 240 also intersects the conical hole 180. It is to be understood, however, that the lower slit 220 and the upper slit 240 are preferably perpendicular with respect to each other and also do not intersect each other. An angle 250 defined by the sides of the upper slit 240 is in the range of about 70-110, preferably about 90 degrees, although other angles could be used, so long as the sides 242 of the upper slit 240 are wider than the angle 185 of the sides 244 of the conical hole 180.

It should be noted that the cross-section shown in FIG. 4B is taken perpendicular to the section shown in FIG. 4A; that is, the lower slit 220 is approximately perpendicular to the upper slit 240.

The perpendicular relationship of the slits 220 and 240 allows the valve 100 to open further to accommodate different and larger sized objects than conventional valves can, but, to still close tightly about the inserted object to reduce or prevent air embolism or fluid leakage. This advantage is achieved by the two non-intersecting slits, one on each of the upper and lower faces 102, 104 of the valve, allowing each section 190, 200 to not move as far as if there were just one slit.

The present invention provides a novel catheter introduce comprising, in one exemplary embodiment, a hub, a catheter sheath, a cap and a valve as described hereinabove. The valve is seated within the hub and cap and, preferably, rotation of the valve within the assembly is prevented by the slots and keys as described.

The present invention has applications beyond the medical device industry. The present invention may be adapted for use in other industries and applications where a valve is used where a leakproof system is needed, and, particularly, where different size objects may be inserted into the valve. Fluid transfer uses can include, but are not limited to, aerospace couplings and lubrication valves, caustic chemical loading and transfer, oil industry pipelines where different diameter pipes must be connected, and the like.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. It should further be noted that any patents, applications and publications referred to herein are incorporated by reference in their entirety.

Claimed is:

1. A sealable valve for a catheter introducer, said valve comprising:
a) a body having an upper surface and a lower surface;
b) a rim having a portion thereof extending beyond said upper surface and a portion thereof extending beyond said lower surface;
c) a first notch formed in said rim portion extending beyond said upper surface;
d) a second notch formed in said rim portion extending beyond said lower surface;
e) a substantially conical depression formed in said body extending from an outer portion proximate to said upper surface and tapering toward an inner portion;
f) an aperture formed in said body coaxial with said conical depression, said aperture having
i) a first portion having a generally conical shape and tapering from said upper surface toward and to generally midway between the center of said conical depression and said lower surface,
ii) a second portion comprising a first slit coaxial with said first portion and extending from said conical depression inner portion into said body,
iii) a third portion comprising a slit flaring outward from and at least partially intersecting said aperture first portion toward said bottom surface;
g) a first thickened portion associated with said bottom surface, said first thickened portion containing at least a portion of said aperture third portion slit; and,
h) a second thickened portion associate with said bottom surface and generally perpendicular to said first thickened portion.

2. The valve of claim 1, wherein said aperture first portion has an angle in the range of about 27 degrees.

3. The valve of claim 1, wherein said conical depression has an angle of about 170 degrees.

4. The valve of claim 1, wherein said aperture first portion has an angle less than the angle of said conical depression.

5. The valve of claim 1, wherein said aperture second portion slit has an angle in the range of about 70-110 degrees.

6. The valve of claim 1, wherein said aperture second portion slit has an angle of about 90 degrees.

7. The valve of claim 1, wherein said aperture third portion slit has an angle of about 90 degrees.

8. The valve of claim 1, wherein said aperture first portion slit and said aperture third portion slit are generally perpendicular to each other.

9. A catheter introducer, comprising:
a) a hub;
b) a catheter sheath;
c) a cap having a tab extending therefrom; and,
d) a valve disposed within said cap, said valve comprising
i) a body having an upper surface and a lower surface,
ii) a rim having a portion thereof extending beyond said upper surface and a portion thereof extending beyond said lower surface,
iii) a first notch formed in said rim portion extending beyond said upper surface,
iv) a second notch formed in said rim portion extending beyond said lower surface, said second notch being matable with said tab,
v) a substantially conical depression formed in said body extending from an outer portion proximate to said upper surface and tapering toward an inner portion,
vi) an aperture formed in said body coaxial with said conical depression, said aperture having
(a) a first portion having a generally conical shape and tapering from said upper surface toward and to generally midway between the center of said conical depression and said lower surface,
(b) a second portion comprising a first slit coaxial with said first portion and extending from said conical depression inner portion into said body,
(c) a third portion comprising a slit flaring outward from and at least partially intersecting said aperture first portion toward said bottom surface,
vii) a first thickened portion associated with said bottom surface, said first thickened portion containing at least a portion of said aperture third portion slit, and
viii) a second thickened portion associate with said bottom surface and generally perpendicular to said first thickened portion.

10. A sealable valve for a catheter introducer, said valve comprising:
a) a body having an upper surface and a lower surface;
b) a rim having a portion thereof extending beyond said upper surface and a portion thereof extending beyond said lower surface;
c) a notch formed in said rim portion extending beyond said lower surface;
d) a substantially conical depression formed in said body extending from an outer portion proximate to said upper surface and tapering toward an inner portion;
e) an aperture formed in said body coaxial with said conical depression, said aperture having
i) a first portion having a generally conical shape and tapering from said upper surface toward and to generally midway between the center of said conical depression and said lower surface,
ii) a second portion comprising a first slit coaxial with said first portion and extending from said conical depression inner portion into said body,
iii) a third portion comprising a slit flaring outward from and at least partially intersecting said aperture first portion toward said bottom surface;
f) a first thickened portion associated with said bottom surface, said first thickened portion containing at least a portion of said aperture third portion slit; and,
g) a second thickened portion associate with said bottom surface and generally perpendicular to said first thickened portion.

11. A sealable valve for a catheter introducer, said valve comprising:
a) a body having an upper surface and a lower surface;
b) a rim having a portion thereof extending beyond said upper surface and a portion thereof extending beyond said lower surface;
c) a substantially conical depression formed in said body extending from an outer portion proximate to said upper surface and tapering toward an inner portion;
d) an aperture formed in said body coaxial with said conical depression, said aperture having
i) a first portion having a generally conical shape and tapering from said upper surface toward and to generally midway between the center of said conical depression and said lower surface,
ii) a second portion comprising a first slit coaxial with said first portion and extending from said conical depression inner portion into said body,
iii) a third portion comprising a slit flaring outward from and at least partially intersecting said aperture first portion toward said bottom surface;
e) a first thickened portion associated with said bottom surface, said first thickened portion containing at least a portion of said aperture third portion slit; and,
f) a second thickened portion associate with said bottom surface and generally perpendicular to said first thickened portion.

12. A sealable valve for a catheter introducer, said valve comprising:
a) a body having an upper surface and a lower surface;
b) a substantially conical depression formed in said body extending from an outer portion proximate to said upper surface and tapering toward an inner portion;
c) an aperture formed in said body coaxial with said conical depression, said aperture having
i) a first portion having a generally conical shape and tapering from said upper surface toward and to generally midway between the center of said conical depression and said lower surface,
ii) a second portion comprising a first slit coaxial with said first portion and extending from said conical depression inner portion into said body,
iii) a third portion comprising a slit flaring outward from and at least partially intersecting said aperture first portion toward said bottom surface;
d) a first thickened portion associated with said bottom surface, said first thickened portion containing at least a portion of said aperture third portion slit; and,
e) a second thickened portion associate with said bottom surface and generally perpendicular to said first thickened portion.

13. A catheter introducer, comprising:
a) a hub;
b) a catheter sheath;
c) a cap having a tab extending therefrom; and,
d) a valve disposed within said cap, said valve comprising
i) a body having an upper surface and a lower surface,
ii) a rim having a portion thereof extending beyond said upper surface and a portion thereof extending beyond said lower surface,
iii) a notch formed in said rim portion extending beyond said lower surface, said notch capable of mating with said tab,
iv) a substantially conical depression formed in said body extending from an outer portion proximate to said upper surface and tapering toward an inner portion,
v) an aperture formed in said body coaxial with said conical depression, said aperture having
(a) a first portion having a generally conical shape and tapering from said upper surface toward and to generally midway between the center of said conical depression and said lower surface,
(b) a second portion comprising a first slit coaxial with said first portion and extending from said conical depression inner portion into said body,
(c) a third portion comprising a slit flaring outward from and at least partially intersecting said aperture first portion toward said bottom surface,
vi) a first thickened portion associated with said bottom surface, said first thickened portion containing at least a portion of said aperture third portion slit, and
vii) a second thickened portion associate with said bottom surface and generally perpendicular to said first thickened portion.

14. A catheter introducer, comprising:
a) a hub;
b) a catheter sheath;
c) a cap; and,
d) a valve disposed within said cap, said valve comprising
i) a body having an upper surface and a lower surface,
ii) a substantially conical depression formed in said body extending from an outer portion proximate to said upper surface and tapering toward an inner portion,
iii) an aperture formed in said body coaxial with said conical depression, said aperture having
(a) a first portion having a generally conical shape and tapering from said upper surface toward and to generally midway between the center of said conical depression and said lower surface,
(b) a second portion comprising a first slit coaxial with said first portion and extending from said conical depression inner portion into said body,
(c) a third portion comprising a slit flaring outward from and at least partially intersecting said aperture first portion toward said bottom surface,
iv) a first thickened portion associated with said bottom surface, said first thickened portion containing at least a portion of said aperture third portion slit, and
v) a second thickened portion associate with said bottom surface and generally perpendicular to said first thickened portion.
